(19) United States
(12) Patent Application Publication
(10) Pub. No.: US 2021/0316080 A1
(43) Pub. Date: Oct. 14, 2021

(54) SYRINGE ASSEMBLY

(71) Applicant: Chung-Sing LIN, Tainan (TW)

(72) Inventor: Chung-Sing LIN, Tainan (TW)

(73) Assignee: Chung-Sing LIN, Tainan (TW)

(21) Appl. No.: 16/842,768

(22) Filed: Apr. 8, 2020

(51) Int. Cl. A61M 5/32

(52) U.S. Cl. CPC A61M 5/3232; A61M 5/3245; A61M 2005/3246; A61M 2005/3256; A61M 2005/3258

(57) ABSTRACT

A syringe assembly includes a main part with a bottom part and a top part. A compressible portion is connected to the top part, and a press unit is formed to the top the compressible portion. A needle is fixed to the top part and located within the compressible portion. The tip of the needle is located corresponding to a film of the press unit. The bottom part has a first lip and a second lip formed to the inner periphery thereof. The top part has a first groove and a second groove in the outer periphery thereof. Multiple collars are telescopically connected to each other, and are connected between the top part and the bottom part. The main part is located in the multiple collars. Each collar has two engaging portions which form two engaging grooves to be engaged with each other when collapsing the collars.

### BACKGROUND OF THE INVENTION

### 1. Fields of the Invention

The present invention relates to a syringe assembly, and more particularly, to a safety syringe assembly whose needle is hidden after being used.

### 2. Descriptions of Related Art

The conventional syringe generally includes a barrel, a plunger and a needle. The needle is installed to the barrel by a locking top on the front end of the barrel, and the plunger is inserted into the barrel from the rear end of the barrel. The medicine in the barrel is ejected into the patient's body via the needle by pushing the plunger toward the needle. When the injection is completed, the user needs to put a cover to the needle to prevent from accessing the user accidently. However, the action of putting the cover to the needle has potential risks, especially under a high tension situation. In addition, how to properly process the used syringes becomes one of concerns of protecting environment.

The present invention intends to provide a syringe assembly to eliminate the shortcomings mentioned above.

### SUMMARY OF THE INVENTION

The present invention relates to a syringe assembly and comprises a main part which is a bellow tube. A top part and a bottom part are respectively formed to the top pend and the lower end of the main part. The bottom part has a protrusion extending therefrom. The protrusion is located within the main part. A first lip and a second lip are formed to the inner periphery of the bottom part. The top part has a passage defined therethrough. A positioning member is formed to the underside of the top part and has a recess which communicates with the interior of the main part. Multiple paths are defined in the underside of the positioning member and communicate with the recess. Two tabs extend from the top part. A first groove and a second groove are respectively defined in the outer periphery of the top part.

A compressible portion is connected to a top of the top part. The compressible portion is a cone-shaped bellow and has a press unit formed to the top thereof. The press unit has a film formed to the center thereof, and two finger flanges extend radially from the press unit. Each finger flange has a hook which hooks the tab corresponding thereto when the compressible potion is compressed and the press unit is moved toward the top part.

A needle is fixed to the passage of the top part and located within the compressible portion. The needle communicates with the recess of the positioning member. The needle has a tip which is located corresponding to the film of the press unit. Multiple collars are telescopically connected to each other and are connected between the top part and the bottom part. The main part is located in the multiple collars. Each collar has two engaging portions, and each engaging portion forms an engaging groove in the opposite side thereof. The engaging groove is sized to be engaged with the engaging portion. The advantages of the present invention are that the needle, the main part and the medicine are combined as a one-piece, so that the users can easily use it, and no cover needs to be mounted to the needle after use. The needle is hidden again after use. The medicine can be injected at desired amount by the telescopically connected collars.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show the syringe assembly of the present invention;

FIG. 2 is a cross sectional view to show the syringe assembly of the present invention;

FIG. 3 illustrates the syringe assembly of the present invention is held by a user;

FIG. 4 is a perspective view to show the syringe assembly of the present invention, wherein the needle penetrates through the film, and two hooks on the finger flanges are shown;

FIG. 5 is a cross sectional view to show that the press unit is pressed to compress the compressible portion;

FIG. 6 is a cross sectional view to show that top one of the collar is engaged with the top part, and the medicine is injected at a desired amount;

FIG. 7 is a perspective view to show that the medicine in the syringe assembly of the present invention is completely injected, and

FIG. 8 is a cross sectional view to show the status of the syringe assembly of the present invention in FIG. 7.

### DETAILED DESCRIPTION OF THE INVENTION

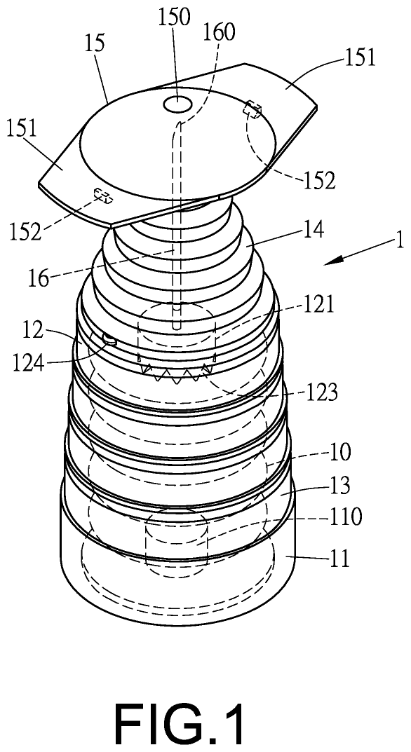

Referring to FIGS. 1 to 5, the syringe assembly 1 of the present invention comprises a main part 10 which is a bellow tube, and a bottom part 11 and a top part 12 are respectively formed to the lower end and the top end of the main part 10. The bottom part 11 has a protrusion 110 extending from the top face thereof, and the protrusion 110 is located within the main part 10. The bottom part 11 further has a first lip 111 and a second lip 112 formed to the inner periphery thereof. The top part 12 has a passage 120 defined therethrough, and a positioning member 121 formed to the underside of the top part 12 and located within the main part 10. The positioning member 121 has a recess 122 which communicates with the interior of the main part 10. Multiple paths 123 are defined in the underside of the positioning member 121 and communicate with the recess 122. Two tabs 124 extend radially from the top part 12. A first groove 125 and a second groove 126 are respectively defined in the outer periphery of the top part 12.

A compressible portion 14 is connected to a top of the top part 12, and the compressible portion 14 is cone-shaped bellow and has a press unit 15 formed to the top thereof. The press unit 15 has a film 150 formed to the center thereof. Two finger flanges 151 extend radially from the press unit 15. Each finger flange 151 has a hook 152 extending from the underside thereof and the hook 152 hooks the tab 124 corresponding thereto when the compressible potion 14 is compressed and the press unit 15 is moved toward the top part 12.

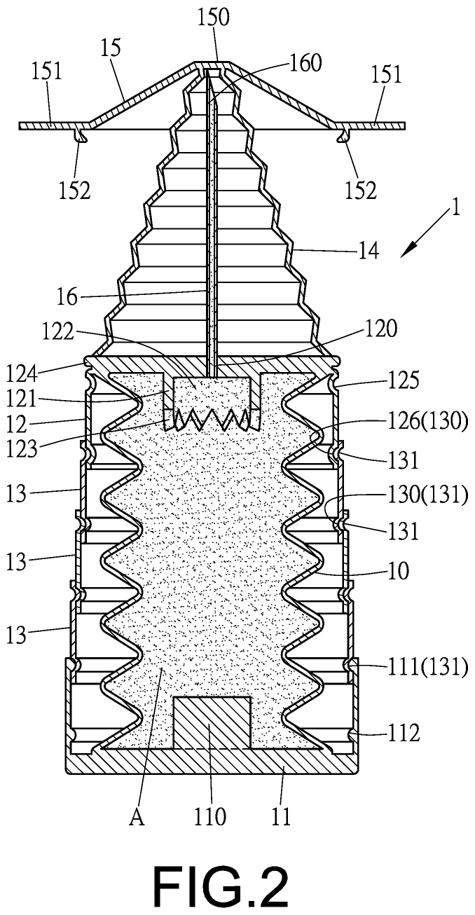

A needle 16 is fixed to the passage 120 of the top part 12 and located within the compressible portion 14. The needle 16 communicates with the recess 122 of the positioning member 121. The needle 16 has a tip 160 which is located corresponding to the film 150 of the press unit 15. Medicine “A” is filled in the main part 10 as shown in FIG. 2. Multiple collars 13 are telescopically connected to each other, and the multiple collars 13 are connected between the top part 12 and the bottom part 11. The main part 10 located in the multiple collars 13. Each collar 13 has two engaging portions 130, and each engaging portion 130 forms an engaging groove 131 in the opposite side thereof. The engaging groove 131 is sized and shaped to be engaged with the engaging portion 130 of the next collar 13.

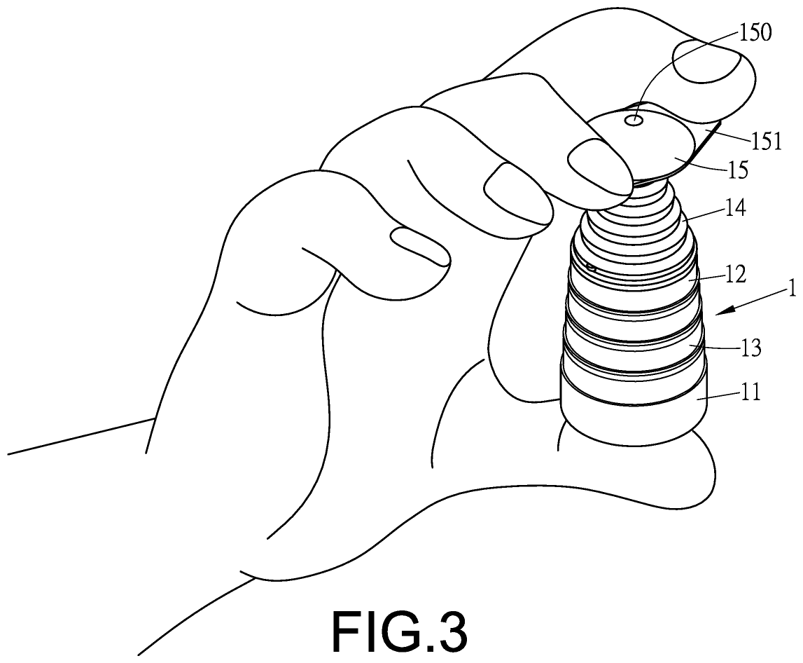

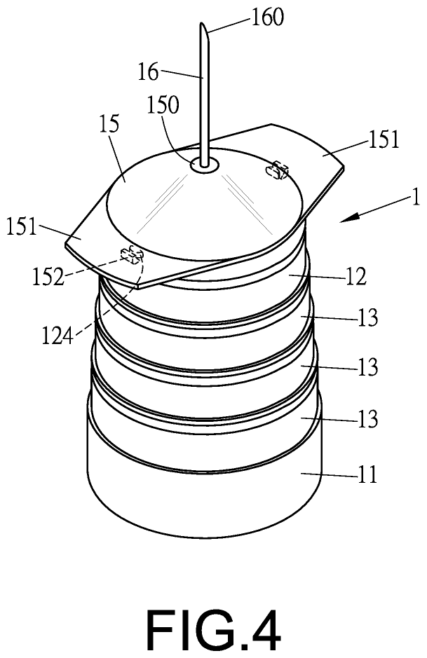

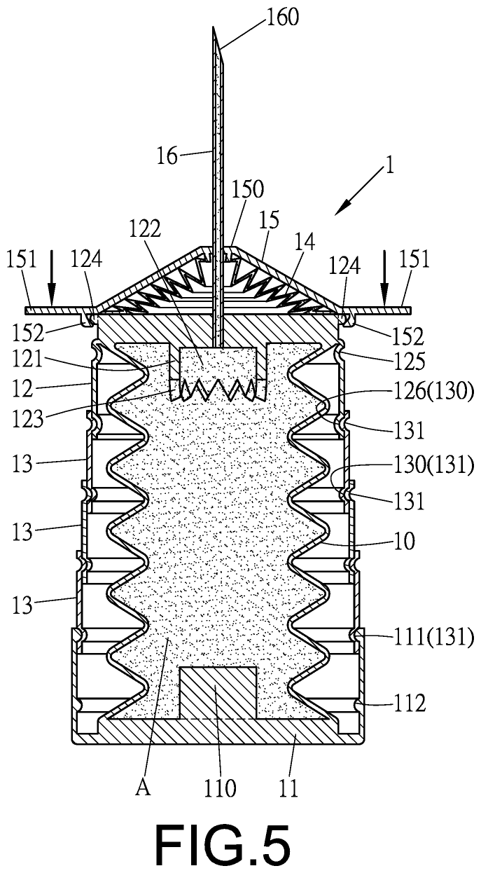

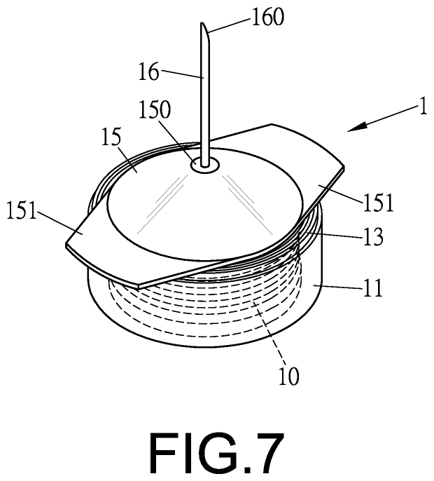

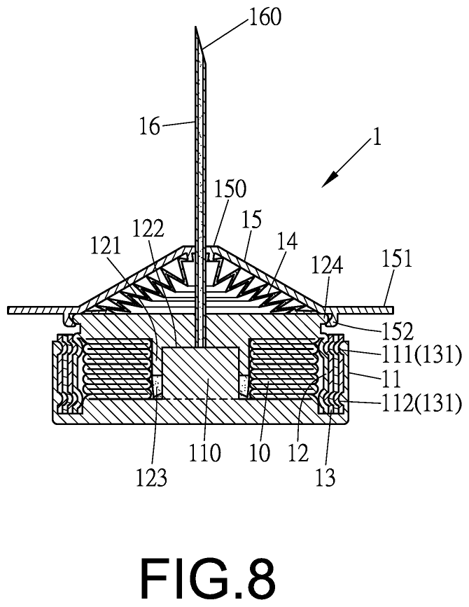

As shown in FIGS. 3 to 8, when in use, the user presses the finger flanges 151 by two fingers and uses the thumb to push the bottom part 11 as shown in FIG. 3. The press unit 15 compresses the compressible portion 14 and moves toward the top part 12. The tip 160 of the needle 16 penetrates through the film 150 of the press unit 15, and the two hooks 152 hook the tabs 124 to secure the press unit 15 to the top part 12 as shown in FIGS. 4 and 5. The needle 16 is then introduced into the patient's limb (not shown), the thumb of the user pushes the main part 10 toward the press unit 15 to main part 10 is compressed to inject the medicine “A” into the patient's body via the recess 122 and the paths 123 as shown in FIGS. 7 and 8. When the protrusion 110 on the bottom part 11 is engaged with the recess 122 of the top part 12, the medicine “A” is completely injected. The main part 10 bounces back to its initial status as shown in FIG. 4 when the user releases the press unit 15 and the bottom part 11. The hooks 152 are then released from the tabs 124, the compressible portion 14 bounces back, and the needle 16 is hidden by the compressible portion 14 and the press unit 15 again so ensure the safety of the user.

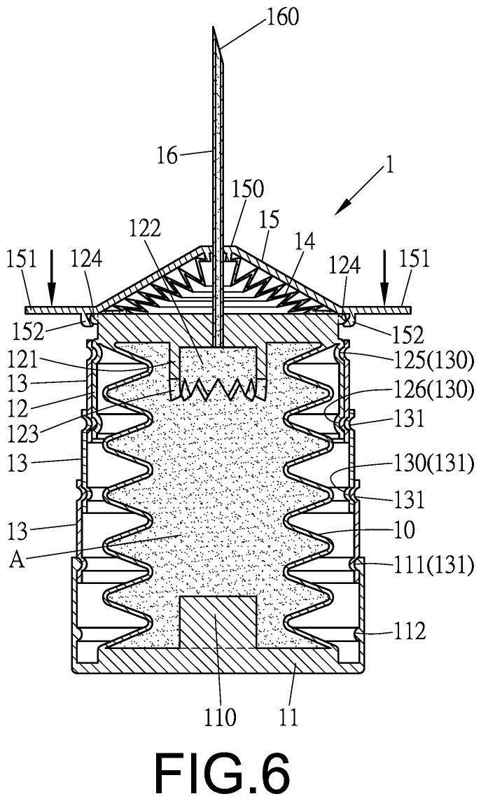

The present invention is able to inject the medicine “A” at desired amount. As shown in FIG. 6, when compressing the press unit 15 and pushing the bottom part 11 to let the top (first) collar 13 to be pushed toward the press unit 15 until the engaging portions 130 of the top (first) collar 13 with the first and second grooves 125, 126 of the top part 12, the first amount (10 ml for example) of the medicine “A” is injected. When continuously pushing the bottom part 11 and the pressing the press unit 15, the second collar 13 is pushed toward the first collar 13, and the next 10 ml medicine “A” is injected. The second collar 13 will be positioned to the outside of the first collar 13, and the engaging portions 130 of the second collar 13 will be engaged with the engaging grooves 131 of the first collar 13. In other words, the user may set the desired amount of the medicine “A” and inject the desired amount of the medicine “A” by engaging the consecutive collars 13. When all of the collars 13 are engaged with each other as shown in FIGS. 7 and 8, the medicine “A” is completely injected.

While we have shown and described the embodiment in accordance with the present invention, it should be clear to those skilled in the art that further embodiments may be made without departing from the scope of the present invention.

What is claimed is:

1. A syringe assembly comprising:
a main part which is a bellow tube, a bottom part formed to a lower end of the main part and having a protrusion extending therefrom and located within the main part, the bottom part having a first lip and a second lip formed to an inner periphery thereof;
a top part formed to a top end of the main part and having a passage defined therethrough, a positioning member formed to an underside of the top part and having a recess which communicates with an interior of the main part, multiple paths defined in an underside of the positioning member and communicating with the recess, two tabs extending from the top part, a first groove and a second groove respectively defined in an outer periphery of the top part;
a compressible portion connected to a top of the top part, the compressible portion being cone-shaped bellow and having a press unit formed to a top thereof, the press unit having a film formed to a center thereof, two finger flanges extending radially from the press unit, each finger flange having a hook which hooks the tab corresponding thereto when the compressible potion is compressed and the press unit is moved toward the top part;
a needle fixed to the passage of the top part and located within the compressible portion, the needle communicating with the recess of the positioning member, the needle having a tip which is located corresponding to the film of the press unit, and
multiple collars telescopically connected to each other, the multiple collars connected between the top part and the bottom part, the main part located in the multiple collars, each collar having two engaging portions, each engaging portion forming an engaging groove in an opposite side thereof, the engaging groove being sized to be engaged with the engaging portion.
